(19) United States
(12) Patent Application Publication
(10) Pub. No.: US 2025/0308629 A1
(43) Pub. Date: Oct. 2, 2025

(54) SMALL VARIANT CALLING WITH ERROR-RATE BASED MODEL

(71) Applicant: GUARDANT HEALTH, INC., Palo Alto, CA (US)

(72) Inventors: Jun ZHAO, San Diego, CA (US); Tingting JIANG, San Diego, CA (US); Marcin Pawel SIKORA, Burlingame, CA (US); Aliaksandr ARTSIOMENKA, Mountain View, CA (US); Rihao QU, South San Francisco, CA (US)

(21) Appl. No.: 19/097,508

(22) Filed: Apr. 1, 2025

Related U.S. Application Data
(60) Provisional application No. 63/572,634, filed on Apr. 1, 2024.

(51) Int. Cl. G16B 20/20; G16B 40/00; G16H 50/30

(52) U.S. Cl. CPC G16B 20/20; G16B 40/00; G16H 50/30

(57) ABSTRACT

Described herein are methods and compositions related to small variant calling Characterizing rare variants implicated in common diseases remains a challenge. Towards these aims, computational efficiency of variant calling have leveraged more advanced computational techniques, including to improve variation detection across more samples or and meet quality control standards for variant calls. Nevertheless, there remains a great need in the art for faster, more effective and accurate variant detection. Here, a small variant calling model based on an error-rate is provided.

### PRIORITY CLAIM

This application claims the benefit of priority to U.S. Provisional Patent Application No. 63/572,634, filed on Apr. 1, 2025, which is incorporated by reference herein in its entirety.

### BACKGROUND

Genetic variants, such as insertions, deletions, substitutions, rearrangements and copy number variants may be correlated with disease and response to therapeutic intervention. Identifying genetic variants accurately is therefore becoming increasingly important for diagnosing and treating disease. Somatic variant calling involves the identification or variants present at low frequency in DNA and is important in the context of cancer treatment. As cancer is caused by an accumulation of DNA mutations in DNA the DNA sample from a tumor is generally heterogeneous, including some normal cells, and cancer cells of different stages. For example, some cells at an early stage of cancer progression, and some late-stage cells. One observes early stage to involves fewer mutations and late stage as involving more mutations. This heterogeneity in sequencing, including the pronounced effects cause by cells of tumor origin can cause somatic mutations will to appear at a low frequency, with a scarce number of sequencing reads covering a given base.

To provide for accurate identification in this complex environment, multiple methods and approaches aim to identify small variants across next-generation sequencing (NGS) short read data. While improvement of methods to identify single-nucleotide variants (SNVs) and small insertions and deletions (indels) from NGS data remains ongoing, there continues to be a need to refine small variant calls, which remain elusive and cannot reliably, accurately and reproducibly identify variant calls in clinical settings. Moreover, characterizing rare variants implicated in common diseases remains a challenge. Towards these aims, computational efficiency of variant calling have leveraged more advanced computational techniques, including to improve variation detection across more samples or and meet quality control standards for variant calls. Nevertheless, there remains a great need in the art for faster, more effective and accurate variant calling methods to efficiently utilize resources in the identification of variations.

### SUMMARY OF THE INVENTION

The disclosure relates detection and analyses of a genetic state of a locus of interest in genetic material. The genetic material may include Deoxyribonucleic Acid (DNA) or Ribonucleic Acid (RNA) from a genome, chromosome, or other genetic material of a sample. The genetic state may include a variation from a wildtype sequence of the nucleic acid sequenced from the sample. Such variation may include, without limitation, a single nucleotide variant (SNV), Indel, nucleic acid rearrangement, and/or other states. Based on the diagnostic, one or more treatment options may be determined. However, other types of genetic states of other loci of interest may be modeled.

Described herein is a method, including accessing sequence information for a plurality of sequence reads generated from a biological sample comprising nucleic acid molecules; identifying a plurality of sequence reads based on a criterion; categorizing each of the plurality of sequence reads into one or more family types; determining an error rate for each of the one or more family types; detecting the presence or absence of a genetic variant in the biological sample based on the determination of error rate of the categorized family type of the plurality of sequence reads. In other embodiments, the error rate is a random error rate, recurrent error rate, or both. In other embodiments, the criterion is an overlap criterion. In other embodiments, the criterion is based on singleton, single strand, double strand criterion. In other embodiments, the criterion is based on strand orientation. In other embodiments, the method includes aligning the plurality of reads to a reference genome; determining one or more loci based on the alignment of the plurality of reads. In other embodiments, the detected genetic variant is at the one or more loci. In other embodiments, the detected genetic variant is a SNV. In other embodiments, the detected genetic variant is an insertion, deletion, and/or nucleic acid rearrangement. In other embodiments, the random error rate is based on one or more of: number of the plurality of sequence reads categorized into one or more family types, strand orientation, strand bias, and nucleotide change. In other embodiments, the recurrent error rate is based on baseline noise from reference samples. In other embodiments, the reference samples are from normal subjects. In other embodiments, the detected genetic variant is based on random error rate, recurrent error rate or both, and further wherein the random error rate is based on one or more of: number of the plurality of sequence reads categorized into one or more family types, strand orientation, strand bias, and nucleotide change and the recurrent error rate is based on baseline noise from reference samples. In other embodiments, the detecting the presence or absence of a genetic variant further comprises a determination based on measurement of one or more of: deamination, read-level error, fragment position, genomic position, hotspot position, mutant allele fraction (MAF), and sequence read diversity. In other embodiments, the method includes determining a predicted disease state based on the detected variant. In other embodiments, the error rate is a random error rate, recurrent error rate, or both, the criterion is one or more of: an overlap criterion, a singleton, a single strand, a double strand criterion and a strand orientation. In various embodiments, the detected genetic variant is a SNV, an insertion, deletion, and/or nucleic acid rearrangement at one or more loci based on the alignment of the plurality of reads. In other embodiments, the error rate is a random error rate, recurrent error rate, or both, the criterion is one or more of: an overlap criterion, a singleton, a single strand, a double strand criterion and a strand orientation. The method of claim 1, wherein the error rate is a random error rate, recurrent error rate, or both, the criterion is one or more of: an overlap criterion, a singleton, a single strand, a double strand criterion and a strand orientation, and the detected genetic variant is a SNV, an insertion, deletion, and/or nucleic acid rearrangement at one or more loci based on the alignment of the plurality of reads. In various embodiments, the error rate is a random error rate, recurrent error rate, or both, the criterion is one or more of: an overlap criterion, a singleton, a single strand, a double strand criterion and a strand orientation, the detected genetic variant is a SNV, an insertion, deletion, and/or nucleic acid rearrangement at one or more loci based on the alignment of the plurality of reads, and further wherein the random error rate is based on one or more of: number of the plurality of sequence reads categorized into one or more family types, strand orientation, strand bias, and nucleotide change, and the recurrent error rate is based on baseline noise from reference samples. In various embodiments, the error rate is a random error rate, recurrent error rate, or both, the criterion is one or more of: an overlap criterion, a singleton, a single strand, a double strand criterion and a strand orientation, the detected genetic variant is a SNV, an insertion, deletion, and/or nucleic acid rearrangement at one or more loci based on the alignment of the plurality of reads, and further wherein the random error rate is based on one or more of: number of the plurality of sequence reads categorized into one or more family types, strand orientation, strand bias, and nucleotide change, the recurrent error rate is based on baseline noise from reference samples, and further wherein the detected genetic variant is based on random error rate, recurrent error rate or both, the random error rate is based on one or more of: number of the plurality of sequence reads categorized into one or more family types, strand orientation, strand bias, and nucleotide change and the recurrent error rate is based on baseline noise from reference samples, and the detecting the presence or absence of a genetic variant further comprises a determination based on measurement of one or more of: deamination, read-level error, fragment position, genomic position, hotspot position, mutant allele fraction (MAF), and sequence read diversity.

Described herein is a method, including accessing sequence information for a plurality of sequence reads generated from a biological sample comprising nucleic acid molecules; identifying a plurality of sequence reads based on a criterion; categorizing each of the plurality of sequence reads into one or more family types; determining an error rate for each of the one or more family types; detecting the presence or absence of a genetic variant in the biological sample based on the determination of error rate of the categorized family type of the plurality of sequence reads. In various embodiments, the biological sample is drawn from liquid, such as blood, plasma, etc. and/or tissue. In various embodiments, the nucleic acid is cell-free DNA. In other embodiments, the method includes filtering plurality of sequence reads based on tiers, a probabilistic model including log likelihood model, genomic and/or hotspot position, mutant allele fraction (MAF). An example is shown in Table 1. In other embodiments, the error rate is a random error rate, recurrent error rate, or both. In other embodiments, the criterion is an overlap criterion. In other embodiments, the criterion is based on singleton, single strand, double strand criterion. In other embodiments, the criterion is based on strand orientation. In various embodiments, identifying a plurality of sequence reads based on a criterion, includes use of a trained machine learning unit. In various embodiments, the method includes identification by the trained machine learning unit, and wherein the trained machine learning unit, wherein the trained machine learning unit is trained by: generating training data, wherein the training data comprises a plurality of sequence reads generated from a training set from training samples of samples drawn from diseased subjects, healthy subjects or both In various embodiments, the plurality of sequence reads are associated with predefined weights, based sequence reads from the different training samples. In various embodiments, the method includes generating a machine learning unit configured to receive input features extracted from the plurality of sequence reads of the training data and generate outputs for each of adenine (A), cytosine (C), guanine (G), and thymine (T) base calls based on the input features, wherein the machine learning unit comprises a neural network or a support vector machine (SVM); and training the machine learning unit with the training data, wherein the training comprises adjusting a set of weights of the neural network or the SVM. In other embodiments, the method includes aligning the plurality of reads to a reference genome; determining one or more loci based on the alignment of the plurality of reads. In other embodiments, the detected genetic variant is at the one or more loci. In other embodiments, the detected genetic variant is a SNV. In other embodiments, the detected genetic variant is an insertion, deletion, and/or nucleic acid rearrangement. In other embodiments, the random error rate is based on one or more of: number of the plurality of sequence reads categorized into one or more family types, strand orientation, strand bias, and nucleotide change. As an example, a random error is approximate to family type and/or the particular nucleotide change. In another example, a random error is approximate to family type, strand properties, and/or the particular nucleotide change. In various embodiments, strand properties include strand bias, which include deamination events (C:G→T:A) and oxidation (C:G→A:T). In various embodiments, related to strand properties, for example, DS has lower error rate than SS, overlap has lower error rate than fwd and rev on most NT changes and DSO-lowest error rate. In various embodiments, pre-filtering criteria include using SNPs from healthy normal samples, removing potential germline, retaining mutants with allele fraction (AF)<1% and estimate error rate using mutant count/total count with a 95% confidence interval. In other embodiments, the recurrent error rate is based on baseline noise from reference samples. In other embodiments, the reference samples are from normal subjects. In other embodiments, the detected genetic variant is based on random error rate, recurrent error rate or both, and further wherein the random error rate is based on one or more of: number of the plurality of sequence reads categorized into one or more family types, strand orientation, strand bias, and nucleotide change and the recurrent error rate is based on baseline noise from reference samples. In other embodiments, the detected genetic variant is based on a log likelihood ratio of error vs. true variant, including Equation 1. In other embodiments, the detected genetic variant is based on one or more of: error rate on double strand (DS), single strand (SS), non-singleton observed AF, and variant score including Equation 1. In other embodiments, the detected genetic variant is based on a baseline noise measurement, including amount of family support and/or removal of germline (e.g., AF>=20%). In other embodiments, the detected genetic variant is based on one or more of: error rate on double strand (DS), single strand (SS), non-singleton observed AF, variant score including Equation 1 and a baseline noise measurement, including amount of family support and/or removal of germline (e.g., AF>=20%). In other embodiments, the method includes filtering plurality of sequence reads based on tiers, a probabilistic model including log likelihood model, genomic and/or hotspot position, mutant allele fraction (MAF). An example is shown in Table 2. In other embodiments, the detecting the presence or absence of a genetic variant further comprises a determination based on measurement of one or more of: deamination, read-level error, fragment position, genomic position, hotspot position, mutant allele fraction (MAF), and sequence read diversity. In other embodiments, the method includes determining a predicted disease state based on the detected variant. In other embodiments, the error rate is a random error rate, recurrent error rate, or both, the criterion is one or more of: an overlap criterion, a singleton, a single strand, a double strand criterion and a strand orientation, and the detected genetic variant is a SNV, an insertion, deletion, and/or nucleic acid rearrangement at one or more loci based on the alignment of the plurality of reads. In other embodiments, the error rate is a random error rate, recurrent error rate, or both, the criterion is one or more of: an overlap criterion, a singleton, a single strand, a double strand criterion and a strand orientation. In other embodiments, the detecting the presence or absence of a genetic variant further comprises generation of one or more error patterns. For example, a filter process can take into account indel support enriched at fragment edges. In various embodiments, the filter process is based on one or more of: distance to a start or end position (e.g., Indel<=10 bases), a molecule count at various distances, and the ratio of molecules counts, such as molecule count with one particular calculated distance in comparison to molecule count without the particular calculated distance. In another example, a filter process can include low diversity in mutant support due to family splitting, with criteria including low diversity in mutant support, being present in SNVs and Indels and present in >1 samples in both normal training and normal from tumor-normal pairs. Additional requirements for filter can include mutant support (e.g., <=10), tags, and based on the support, tags, determining a set, the detection of the genetic variant is based on the number of determined sets.

A system configured to perform any of the aforementioned methods.

A computer readable medium, comprising instructions for performing any of the aforementioned methods.

### BRIEF DESCRIPTION OF THE FIGURES

FIG. 1. Overview of family grouping and counting for variant candidates.

FIG. 2. Main challenge of small variant calling is to identify non-ref loci and distinguish true mutation from error. This error can include recurrent error, such as alignment bias or sequencing artifact in low complexity regions and random error, such as deamination or oxidation caused by DNA damage.

FIG. 3. SNV calling. Error rate profile of different family types and NT changes.

FIG. 4. SNV calling. error rate profiles of different panels and materials in FIG. 4A, liquid (n=276) double stranded (DS) & single stranded (SS) was characterized by high C>T/G>A, low A>T/T>A. In contrived samples (n=59), the overlap is similar to fwd/rev DS not much lower than SS to constitute a material difference. In FIG. 4b,iIn tissue healthy samples (n=124), there was a higher error rate than liquid, higher sample variation and high DNA damage related errors.

FIG. 5. SNV fragment end filter. An additional fragment end filter eliminates false positives (FPs). Here, one compares distribution of relative distance in mut and ref support. One then removes FPs with mutant support clustered at fragment end/start, including FIG. 5a and FIG. 5b.

FIG. 6. Small variant calling in Tissue vs. Liquid. Here, a challenge is the lower diversity and lower double strand ratio in tissue than liquid. In some instances, one may opt to not sequence to saturation to maintain low sequencing resources, leading to a high proportion of singletons. A mitigation strategy includes adding singletons into consideration for small variant calling.

FIG. 7. Comparison of SNV error rate in Tissue vs. Liquid. In tissue, an exemplary error rate range is 1e-6˜1e-3, which potential inflation due to low quality normal training samples, with a higher error rate as a conservative option. In liquid methylation detection, an exemplary error rate range is 1e-8˜1e-4.

FIG. 8. Re-training removes majority of low mutant allele fraction (MAF) variants in normal samples Most residual variants are below 1%, which can be remove with a MAF filter. This allows identification of new error patterns for false positives >=1%

FIG. 9. Error pattern. As shown, the aforementioned methods identifies indel support being enriched at fragment edge, which is present in both deletion and insertion. This further includes being present in >1 samples in both normal training and normal from tumor-normal pairs. When implementing the additional filter described above, one defines define bad distance when Indel is <=10 bases from fragment start or end, followed by count ratio of mutant support molecules with bad distance and fail Indel call if ratio >0.66.

FIG. 10. Additional error pattern. Low diversity in mutant support due to family splitting. For example low diversity in mutant support, present in SNVs and Indels, Present in >1 samples in both normal training and normal from tumor-normal pairs. When implementing the additional filter described above, criteria include mutant support <=10, creation of unique sets for mutant MK tags, followed by determination of the size of each set, fail variant call if <2 sets with size >1.

### DETAILED DESCRIPTION

### Computer Implementation and Analysis Pipeline

The present methods can be computer-implemented, such that any or all of the steps described in the specification or appended claims other than wet chemistry steps can be performed in a suitable programmed computer. The computer can be a mainframe, personal computer, tablet, smart phone, cloud, online data storage, remote data storage, or the like. The computer can be operated in one or more locations.

Various operations of the present methods can utilize information and/or programs and generate results that are stored on computer-readable media (e.g., hard drive, auxiliary memory, external memory, server; database, portable memory device (e.g., CD-R, DVD, ZIP disk, flash memory cards), and the like.

The present disclosure also includes an article of manufacture for analyzing a nucleic acid population that includes a machine-readable medium containing one or more programs which when executed implement the steps of the present methods.

The disclosure can be implemented in hardware and/or software. For example, different aspects of the disclosure can be implemented in either client-side logic or server-side logic. The disclosure or components thereof can be embodied in a fixed media program component containing logic instructions and/or data that when loaded into an appropriately configured computing device cause that device to perform according to the disclosure. A fixed media containing logic instructions can be delivered to a viewer on a fixed media for physically loading into a viewer's computer or a fixed media containing logic instructions may reside on a remote server that a viewer accesses through a communication medium to download a program component.

The code may be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible, storage media, “media” may include other types of (intangible) media.

“Storage” media, terms such as computer or machine “readable medium” refer to any tangible (such as physical), non-transitory, medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, a report. Examples of UI's include, without limitation, a graphical user interface (GUI) and web-based user interface.

Methods and systems of the present disclosure can be implemented by way of one or more algorithms. An algorithm can be implemented by way of software upon execution by the processor.

In some embodiments, the system is a computer system that may include a processor programmed to access a plurality of paired-end reads generated from the sample of nucleic acid molecules from the subject, identify a plurality of pairs of sequence reads from among the plurality of sequence reads based on an overlap criterion, tags, strand orientation etc. In some embodiments, detection of a genetic variant is based on the plurality of pairs of overlapping sequence reads, including categorization into a family. This may include a sequence based on respective sequences of a pair of overlapping sequence reads. The processor may be further programmed to identify sequence read that does not satisfy an overlap criterion with another sequence read based on other criterion. The processor may be further programmed to align the plurality of sequence reads to a reference genome to generate a plurality of aligned reads, identify a plurality of genetic loci for each of the plurality of aligned reads. In some embodiments, one may cluster the plurality of sequence reads based on characteristics of the sequence read itself (e.g., distance from start or end, strand orientation) and/or a sub-sequence or the sequence read.

In some embodiments, the system may further include a laboratory system to amplify polynucleotides from the sample of the subject. In some embodiments, the processor may be further programmed to determine that the detected variant comprises an insertion, a deletion, or a nucleic acid rearrangement. In some embodiments, the processor may be further programmed to determine a predicted disease state based on the detected variant.

In some embodiments, a system includes accessing sequence information for a plurality of sequence reads generated from a biological sample comprising nucleic acid molecules; identifying a plurality of sequence reads based on a criterion; categorizing each of the plurality of sequence reads into one or more family types; determining an error rate for each of the one or more family types; detecting the presence or absence of a genetic variant in the biological sample based on the determination of error rate of the categorized family type of the plurality of sequence reads. In various embodiments, the biological sample is drawn from liquid, such as blood, plasma, etc. and/or tissue. In various embodiments, the nucleic acid is cell-free DNA. In other embodiments, the method includes filtering plurality of sequence reads based on tiers, a probabilistic model including log likelihood model, genomic and/or hotspot position, mutant allele fraction (MAF). An example is shown in Table 1. In other embodiments, the error rate is a random error rate, recurrent error rate, or both. In other embodiments, the criterion is an overlap criterion. In other embodiments, the criterion is based on singleton, single strand, double strand criterion. In other embodiments, the criterion is based on strand orientation. In various embodiments, identifying a plurality of sequence reads based on a criterion, includes use of a trained machine learning unit. In various embodiments, the method includes identification by the trained machine learning unit, and wherein the trained machine learning unit, wherein the trained machine learning unit is trained by: generating training data, wherein the training data comprises a plurality of sequence reads generated from a training set from training samples of samples drawn from diseased subjects, healthy subjects or both In various embodiments, the plurality of sequence reads are associated with predefined weights, based sequence reads from the different training samples. In various embodiments, the method includes generating a machine learning unit configured to receive input features extracted from the plurality of sequence reads of the training data and generate outputs for each of adenine (A), cytosine (C), guanine (G), and thymine (T) base calls based on the input features, wherein the machine learning unit comprises a neural network or a support vector machine (SVM); and training the machine learning unit with the training data, wherein the training comprises adjusting a set of weights of the neural network or the SVM. In other embodiments, the method includes aligning the plurality of reads to a reference genome; determining one or more loci based on the alignment of the plurality of reads. In other embodiments, the detected genetic variant is at the one or more loci. In other embodiments, the detected genetic variant is a SNV. In other embodiments, the detected genetic variant is an insertion, deletion, and/or nucleic acid rearrangement. In other embodiments, the random error rate is based on one or more of: number of the plurality of sequence reads categorized into one or more family types, strand orientation, strand bias, and nucleotide change. As an example, a random error is approximate to family type and/or the particular nucleotide change. In another example, a random error is approximate to family type, strand properties, and/or the particular nucleotide change. In various embodiments, strand properties include strand bias, which include deamination events (C:G→T:A) and oxidation (C:G→A:T). In various embodiments, related to strand properties, for example, DS has lower error rate than SS, overlap has lower error rate than fwd and rev on most NT changes and DSO-lowest error rate. In various embodiments, pre-filtering criteria include using SNPs from healthy normal samples, removing potential germline, retaining mutants with allele fraction (AF)<1% and estimate error rate using mutant count/total count with a 95% confidence interval. In other embodiments, the recurrent error rate is based on baseline noise from reference samples. In other embodiments, the reference samples are from normal subjects. In other embodiments, the detected genetic variant is based on random error rate, recurrent error rate or both, and further wherein the random error rate is based on one or more of: number of the plurality of sequence reads categorized into one or more family types, strand orientation, strand bias, and nucleotide change and the recurrent error rate is based on baseline noise from reference samples. In other embodiments, the detected genetic variant is based on a log likelihood ratio of error vs. true variant, including Equation 1 or other probabilistic model. In other embodiments, the detected genetic variant is based on one or more of: error rate on double strand (DS), single strand (SS), non-singleton observed AF, and variant score including Equation 1 or other probabilistic model. In other embodiments, the detected genetic variant is based on a baseline noise measurement, including amount of family support and/or removal of germline (e.g., AF>=20%). In other embodiments, the detected genetic variant is based on one or more of: error rate on double strand (DS), single strand (SS), non-singleton observed AF, variant score including Equation 1 and a baseline noise measurement, including amount of family support and/or removal of germline (e.g., AF>=20%). In other embodiments, the method includes filtering plurality of sequence reads based on tiers, a probabilistic model including log likelihood model, genomic and/or hotspot position, mutant allele fraction (MAF). An example is shown in Table 2. In other embodiments, the detecting the presence or absence of a genetic variant further comprises a determination based on measurement of one or more of: deamination, read-level error, fragment position, genomic position, hotspot position, mutant allele fraction (MAF), and sequence read diversity. In other embodiments, the method includes determining a predicted disease state based on the detected variant. In other embodiments, the error rate is a random error rate, recurrent error rate, or both, the criterion is one or more of: an overlap criterion, a singleton, a single strand, a double strand criterion and a strand orientation, and the detected genetic variant is a SNV, an insertion, deletion, and/or nucleic acid rearrangement at one or more loci based on the alignment of the plurality of reads. In other embodiments, the error rate is a random error rate, recurrent error rate, or both, the criterion is one or more of: an overlap criterion, a singleton, a single strand, a double strand criterion and a strand orientation. In other embodiments, the detecting the presence or absence of a genetic variant further comprises generation of one or more error patterns. For example, a filter process can take into account indel support enriched at fragment edges. In various embodiments, the filter process is based on one or more of: distance to a start or end position (e.g., Indel <=10 bases), a molecule count at various distances, and the ratio of molecules counts, such as molecule count with one particular calculated distance in comparison to molecule count without the particular calculated distance. In another example, a filter process can include low diversity in mutant support due to family splitting, with criteria including low diversity in mutant support, being present in SNVs and Indels and present in >1 samples in both normal training and normal from tumor-normal pairs. Additional requirements for filter can include mutant support (e.g., <=10), tags, and based on the support, tags, determining a set, the detection of the genetic variant is based on the number of determined sets.

The various steps of the methods disclosed herein, or the steps carried out by the systems disclosed herein, may be carried out at the same time or different times, and/or in the same geographical location or different geographical locations, e.g. countries. The various steps of the methods disclosed herein can be performed by the same person or different people.

### Sample Collection

A sample may be any biological sample isolated from a subject. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. Such samples include nucleic acids shed from tumors. The nucleic acids can include DNA and RNA and can be in double- and/or single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, for example, a body fluid for analysis is plasma or serum containing cell-free nucleic acids, e.g., cell-free DNA (cfDNA).

In certain implementations, the polynucleotides can be enriched prior to sequencing. Enrichment can be performed for specific genomic target sequences. In certain embodiments, the specific genomic target sequences do not include the locus of interest. For example, the specific genomic target sequences may not include any portion of the locus of interest. In certain other implementations, enrichment can be performed nonspecifically. In some implementations, targeted regions of interest may be enriched with capture probes (“baits”) selected for one or more bait set panels using a differential tiling and capture scheme. A differential tiling and capture scheme uses bait sets of different relative concentrations to differentially tile (e.g., at different “resolutions”) across genomic regions associated with baits, subject to a set of constraints (e.g., sequencer constraints such as sequencing load, utility of each bait, etc.), and capture them at a desired level for downstream sequencing. These targeted genomic regions of interest may include regions of a subject's genome or transcriptome. In some implementations, biotin-labeled beads with probes to one or more regions of interest can be used to capture target sequences, optionally followed by amplification of those regions, to enrich for the regions of interest.

Sequence capture typically involves the use of oligonucleotide probes that hybridize to the target sequence. A probe set strategy can involve tiling the probes across a region of interest. Such probes can be, e.g., about 60 to 130 bases long. The set can have a depth of about 2×, 3×, 4×, 5×, 6×, 8×, 9×, 10×, 15×, 30×, 50×, or more. The effectiveness of sequence capture depends, in part, on the length of the sequence in the target molecule that is complementary (or nearly complementary) to the sequence of the probe.

In some implementations, the methods of the disclosure comprise selectively enriching regions from the subject's genome or transcriptome prior to sequencing. In other implementations, the methods of the disclosure comprise non-selectively enriching regions from the subject's genome or transcriptome prior to sequencing.

In certain implementations, sample index sequences are introduced to the polynucleotides after enrichment. The sample index sequences may be introduced through PCR or ligated to the polynucleotides, optionally as part of adapters.

The volume of plasma can depend on the desired read depth for sequenced regions. Exemplary volumes are 0.4-40 ml, 5-20 ml, 10-20 ml. For example, the volume can be 0.5 ml, 1 ml, 5 ml, 10 ml, 20 ml, 30 ml, or 40 ml. A volume of sampled plasma may be 5 to 20 ml.

The sample can comprise various amounts of nucleic acid that contains genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell free. A sample can comprise nucleic acids carrying mutations. For example, a sample can comprise DNA carrying germline mutations and/or somatic mutations. A sample can comprise DNA carrying cancer-associated mutations (e.g., cancer-associated somatic mutations).

Exemplary amounts of cell free nucleic acids in a sample before amplification range from about 1 fg to about 1 μg, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

A cell-free nucleic acid sample refers to a sample containing cell-free nucleic acids. In some embodiments, “cell-free nucleic acids” refers to nucleic acids not contained within or otherwise bound to a cell at the point of isolation from the subject. Cell-free nucleic acids can be referred to all non-encapsulated nucleic acid sourced from a bodily fluid (e.g., blood, urine, CSF, etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), RNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA (ctDNA). Others are released from healthy cells. ctDNA can be non-encapsulated tumor-derived fragmented DNA. Cell-free fetal DNA (cffDNA) is fetal DNA circulating freely in the maternal blood stream.

A cell-free nucleic acid or proteins associated with it can have one or more epigenetic modifications, for example, a cell-free nucleic acid can be acetylated, 5-methylated, ubiquitylated, phosphorylated, sumoylated, ribosylated, and/or citrullinated.

Cell-free nucleic acids have an exemplary size distribution of about 100-500 nucleotides, with molecules of 110 to about 230 nucleotides representing about 90% of molecules, with a mode of about 168 nucleotides in humans and a second minor peak in a range between 240 to 430 nucleotides. Cell-free nucleic acids can be about 160 to about 180 nucleotides, or about 320 to about 360 nucleotides, or about 430 to about 480 nucleotides.

Cell-free nucleic acids can be isolated from bodily fluids through a partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, cell-free nucleic acids can be precipitated with an alcohol. Further clean up steps may be used such as silica based columns to remove contaminants or salts. Non-specific bulk carrier nucleic acids, for example, may be added throughout the reaction to optimize certain aspects of the procedure such as yield.

After such processing, samples can include various forms of nucleic acid including double-stranded DNA, single stranded DNA and single stranded RNA. Optionally, single stranded DNA and RNA can be converted to double-stranded forms so they are included in subsequent processing and analysis steps.

### Amplification

Sample nucleic acids flanked by adapters can be amplified by PCR and other amplification methods typically primed from primers binding to primer binding sites in adapters flanking a DNA molecule to be amplified. Amplification methods can involve cycles of extension, denaturation and annealing resulting from thermocycling or can be isothermal as in transcription mediated amplification. Other amplification methods include the ligase chain reaction, strand displacement amplification, nucleic acid sequence based amplification, and self-sustained sequence based replication.

One or more amplifications can be applied to introduce barcodes to a nucleic acid molecule using conventional nucleic acid amplification methods. The amplification can be conducted in one or more reaction mixtures. Molecular barcodes and sample indexes can be introduced simultaneously, or in any sequential order. Molecular barcodes and sample indexes can be introduced prior to and/or after sequence capturing. In some cases, only the molecular barcodes are introduced prior to probe capturing while the sample indexes are introduced after sequence capturing. In some cases, both the molecular barcodes and the sample indexes are introduced prior to probe capturing. In some cases, the sample indexes are introduced after sequence capturing. Usually, sequence capturing involves introducing a single-stranded nucleic acid molecule complementary to a targeted sequence, e.g., a coding sequence of a genomic region and mutation of such region is associated with a cancer type. Typically, the amplifications generate a plurality of non-uniquely or uniquely tagged nucleic acid amplicons with molecular barcodes and sample indexes at a size ranging from 200 nt to 700 nt, 250 nt to 350 nt, or 320 nt to 550 nt. In some implementations, the amplicons have a size of about 300 nt. In some implementations, the amplicons have a size of about 500 nt.

### Barcodes

Barcodes can be incorporated into or otherwise joined to adapters by chemical synthesis, ligation, overlap extension PCR among other methods. Generally, assignment of unique or non-unique barcodes in reactions follows methods and systems described by U.S. patent applications 20010053519, 20110160078, and U.S. Pat. Nos. 6,582,908 and 7,537,898 and 9,598,731.

Tags can be linked to sample nucleic acids randomly or non-randomly. In some cases, they are introduced at an expected ratio of identifiers (e.g., a combination of barcodes) to microwells. The collection of barcodes can be unique, e.g., all the barcodes have different nucleotide sequence. The collection of barcodes can be non-unique, e.g., some of the barcodes have the same nucleotide sequence, and some of the barcodes have different nucleotide sequence. For example, the identifiers may be loaded so that more than 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some cases, the identifiers may be loaded so that less than 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers are loaded per genome sample. In some cases, the average number of identifiers loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 identifiers per genome sample.

A preferred format uses 20-50 different tags, ligated to both ends of a target molecule creating 20-50×20-50 tags, e.g., 400-2500 tags combinations. Such numbers of tags are sufficient that different molecules having the same start and stop points have a high probability (e.g., at least 94%, 99.5%, 99.99%, 99.999%) of receiving different combinations of tags.

In some cases, identifiers may be predetermined or random or semi-random sequence oligonucleotides. In other cases, a plurality of barcodes may be used such that barcodes are not necessarily unique to one another in the plurality. In this example, barcodes may be attached (e.g., by ligation or PCR amplification) to individual molecules such that the combination of the barcode and the sequence it may be attached to creates a unique sequence that may be individually tracked. As described herein, detection of non-uniquely tagged barcodes in combination with sequence data of beginning (start) and end (stop) positions of sequence reads may allow assignment of a unique identity to a particular molecule. The length, or number of base pairs, of an individual sequence read may also be used to assign a unique identity to such a molecule. As described herein, fragments from a single strand of nucleic acid having been assigned a unique identity, may thereby permit subsequent identification of fragments from the parent strand, and/or a complementary strand.

### Sequencing Pipeline

Sample nucleic acids flanked by adapters with or without prior amplification can be subject to sequencing, such as by one or more sequencing devices 107. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLID, Ion Torrent, or Nanopore platforms. Sequencing reactions can be performed in a variety of sample processing units, which may be multiple lanes, multiple channels, multiple wells, or other means of processing multiple sample sets substantially simultaneously. Sample processing unit can also include multiple sample chambers to enable processing of multiple runs simultaneously.

The sequencing reactions can be performed on one or more fragments types known to contain markers of cancer of other disease. The sequencing reactions can also be performed on any nucleic acid fragments present in the sample. The sequence reactions may provide for sequence coverage of the genome of at least 5%, 10%, 15%, 20%, 25%, 30%, 40%, 50%, 60%, 70%, 80%, 90%, 95%, 99%, 99.9% or 100%. In other cases, sequence coverage of the genome may be less than 5%, 10%, 15%, 20%, 25%, 30%, 40%, 50%, 60%, 70%, 80%, 90%, 95%, 99%, 99.9% or 100%.

Simultaneous sequencing reactions may be performed using multiplex sequencing. In some cases, cell free polynucleotides may be sequenced with at least 1000, 2000, 3000, 4000, 5000, 6000, 7000, 8000, 9000, 10000, 50000, 100,000 sequencing reactions. In other cases, cell free polynucleotides may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

### Sequence Analysis Pipeline

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition.

Various cancers may be detected using the present methods. Cancers cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as rare mutations. This phenomenon may be used to detect the presence or absence of cancers individuals using the methods and systems described herein.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors, and the like.

Cancers can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers progress, becoming more aggressive and genetically unstable. Other cancers may remain benign, inactive, or dormant. The system and methods of this disclosure may be useful in determining disease progression.

The present analysis is also useful in determining the efficacy of a particular treatment option. Successful treatment options may increase the amount of copy number variation or rare mutations detected in a subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens are changing during the course of infection. This may be particularly important during chronic infections, such as HIV/AIDS or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

### Determination of 5-Methylcytosine Pattern of Nucleic Acids

Bisulfite-based sequencing and variants thereof provides a means of determining the methylation pattern of a nucleic acid. In some embodiments, determining the methylation pattern includes distinguishing 5-methylcytosine (5mC) from non-methylated cytosine. In some embodiments, determining methylation pattern includes distinguishing N6-methyladenine from non-methylated adenine. In some embodiments, determining the methylation pattern includes distinguishing 5-hydroxymethylcytosine (5hmC), 5-formylcytosine (5fC), and 5-carboxylcytosine (5caC) from non-methylated cytosine. Examples of bisulfite sequencing include, but are not limited to oxidative bisulfite sequencing (OX-BS-seq), Tet-assisted bisulfite sequencing (TAB-seq), and reduced bisulfite sequencing (redBS-seq).

Oxidative bisulfite sequencing (OX-BS-seq) is used to distinguish between 5mC and 5hmC, by first converting the 5hmC to 5fC, and then proceeding with bisulfite sequencing as previously described. Tet-assisted bisulfite sequencing (TAB-seq) can also be used to distinguish 5mc and 5hmC. In TAB-seq, 5hmC is protected by glucosylation. A Tet enzyme is then used to convert 5mC to 5caC before proceeding with bisulfite sequencing, as previously described. Reduced bisulfite sequencing is used to distinguish 5fC from modified cytosines.

Generally, in bisulfite sequencing, a nucleic acid sample is divided into two aliquots and one aliquot is treated with bisulfite. The bisulfite converts native cytosine and certain modified cytosine nucleotides (e.g. 5-formylcytosine or 5-carboxylcytosine) to uracil whereas other modified cytosines (e.g., 5-methylcytosine, 5-hydroxylmethylcystosine) are not converted. Comparison of nucleic acid sequences of molecules from the two aliquots indicates which cytosines were and were not converted to uracils. Consequently, cytosines which were and were not modified can be determined. The initial splitting of the sample into two aliquots is disadvantageous for samples containing only small amounts of nucleic acids, and/or composed of heterogeneous cell/tissue origins such as bodily fluids containing cell-free DNA.

The present disclosure provides methods allowing bisulfite sequencing and variants thereof. These methods work by linking nucleic acids in a population to a capture moiety, i.e., a label that can be captured or immobilized. Capture moieties include, without limitation, biotin, avidin, streptavidin, a nucleic acid including a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase. Following linking of capture moieties to sample nucleic acids, the sample nucleic acids serve as templates for amplification. Following amplification, the original templates remain linked to the capture moieties, but amplicons are not linked to capture moieties.

The capture moiety can be linked to sample nucleic acids as a component of an adapter, which may also provide amplification and/or sequencing primer binding sites. In some methods, sample nucleic acids are linked to adapters at both ends, with both adapters bearing a capture moiety. Preferably any cytosine residues in the adapters are modified, such as by 5methylcytosine, to protect against the action of bisulfite. In some instances, the capture moieties are linked to the original templates by a cleavable linkage (e.g., photocleavable desthiobiotin-TEG or uracil residues cleavable with USER™ enzyme, Chem. Commun. (Camb). 2015 Feb. 21; 51 (15): 3266-3269), in which case the capture moieties can, if desired, be removed.

The amplicons are denatured and contacted with an affinity reagent for the capture tag. Original templates bind to the affinity reagent whereas nucleic acid molecules resulting from amplification do not. Thus, the original templates can be separated from nucleic acid molecules resulting from amplification.

Following separation or partition, the respective populations of nucleic acids (i.e., original templates and amplification products) can be subjected to bisulfite treatment with the original template population receiving bisulfite treatment and the amplification products not. Alternatively, the amplification products can be subjected to bisulfite treatment and the original template population is not. Following such treatment, the respective populations can be amplified (which in the case of the original template population converts uracils to thymines). The populations can also be subjected to biotin probe hybridization for enrichment. The respective populations are then analyzed and sequences compared to determine which cytosines were 5-methylated (or 5-hydroxylmethylated) in the original. Detection of a T nucleotide in the template population (corresponding to an unmethylated cytosine converted to uracil) and a C nucleotide at the corresponding position of the amplified population indicates an unmodified C. The presence of C's at corresponding positions of the original template and amplified populations indicates a modified C in the original sample.

In some embodiments, a method uses sequential DNA-seq and bisulfite-seq (BIS-seq) NGS library preparation of molecular tagged DNA libraries. This process is performed by labeling of adapters (e.g., biotin), DNA-seq amplification of whole library, parent molecule recovery (e.g. streptavidin bead pull down), bisulfite conversion and BIS-seq. In some embodiments, the method identifies 5-methylcytosine with single-base resolution, through sequential NGS-preparative amplification of parent library molecules with and without bisulfite treatment. This can be achieved by modifying the 5-methyl-ated NGS-adapters (directional adapters; Y-shaped/forked with 5-methylcytosine replacing) used in BIS-seq with a label (e.g., biotin) on one of the two adapter strands. Sample DNA molecules are adapter ligated, and amplified (e.g., by PCR). As only the parent molecules will have a labeled adapter end, they can be selectively recovered from their amplified progeny by label-specific capture methods (e.g., streptavidin-magnetic beads). As the parent molecules retain 5-methylation marks, bisulfite conversion on the captured library will yield single-base resolution 5-methylation status upon BIS-seq, retaining molecular information to corresponding DNA-seq. In some embodiments, the bisulfite treated library can be combined with a non-treated library prior to enrichment/NGS by addition of a sample tag DNA sequence in standard multiplexed NGS workflow. As with BIS-seq workflows, bioinformatics analysis can be carried out for genomic alignment and 5-methylated base identification. In sum, this method provides the ability to selectively recover the parent, ligated molecules, carrying 5-methylcytosine marks, after library amplification, thereby allowing for parallel processing for bisulfite converted DNA. This overcomes the destructive nature of bisulfite treatment on the quality/sensitivity of the DNA-seq information extracted from a workflow. With this method, the recovered ligated, parent DNA molecules (via labeled adapters) allow amplification of the complete DNA library and parallel application of treatments that elicit epigenetic DNA modifications. The present disclosure discusses the use of BIS-seq methods to identify cytosine5-methylation (5-methylcytosine), but this is not limiting. Variants of BIS-seq have been developed to identify hydroxymethylated cytosines (5hmC; OX-BS-seq, TAB-seq), formylcytosine (5fC; redBS-seq) and carboxylcytosines. These methodologies can be implemented with the sequential/parallel library preparation described herein.

### Alternative Methods of Modified Nucleic Acid Analysis

The disclosure provides alternative methods for analyzing modified nucleic acids (e.g., methylated, linked to histones and other modifications discussed above). In some such methods, a population of nucleic acids bearing the modification to different extents (e.g., 0, 1, 2, 3, 4, 5 or more methyl groups per nucleic acid molecule) is contacted with adapters before fractionation of the population depending on the extent of the modification. Adapters attach to either one end or both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. Following attachment of adapters, the nucleic acids are amplified from primers binding to the primer binding sites within the adapters. Adapters, whether bearing the same or different tags, can include the same or different primer binding sites, but preferably adapters include the same primer binding site. Following amplification, the nucleic acids are contacted with an agent that preferably binds to nucleic acids bearing the modification (such as the previously described such agents). The nucleic acids are separated into at least two partitions differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. Following separation, the different partitions can then be subject to further processing steps, which typically include further amplification, and sequence analysis, in parallel but separately. Sequence data from the different partitions can then be compared.

Nucleic acids can be linked at both ends to Y-shaped adapters including primer binding sites and tags. The molecules are amplified. The amplified molecules are then fractionated by contact with an antibody preferentially binding to 5-methylcytosine to produce two partitions. One partition includes original molecules lacking methylation and amplification copies having lost methylation. The other partition includes original DNA molecules with methylation. The two partitions are then processed and sequenced separately with further amplification of the methylated partition. The sequence data of the two partitions can then be compared. In this example, tags are not used to distinguish between methylated and unmethylated DNA but rather to distinguish between different molecules within these partitions so that one can determine whether reads with the same start and stop points are based on the same or different molecules.

The disclosure provides further methods for analyzing a population of nucleic acid in which at least some of the nucleic acids include one or more modified cytosine residues, such as 5-methylcytosine and any of the other modifications described previously. In these methods, the population of nucleic acids is contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are treated with bisulfite. This treatment converts unmodified cytosines to uracils. The bisulfite treated nucleic acids are then subjected to amplification primed by primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Partitioning the Sample into a Plurality of Subsamples; Aspects of Samples; Analysis of Epigenetic Characteristics

In certain embodiments described herein, a population of different forms of nucleic acids (e.g., hypermethylated and hypomethylated DNA in a sample, such as a captured set of cfDNA as described herein) can be physically partitioned based on one or more characteristics of the nucleic acids prior to further analysis, e.g., differentially modifying or isolating a nucleobase, tagging, and/or sequencing. This approach can be used to determine, for example, whether certain sequences are hypermethylated or hypomethylated. In some embodiments, hypermethylation variable epigenetic target regions are analyzed to determine whether they show hypermethylation characteristic of tumor cells and/or hypomethylation variable epigenetic target regions are analyzed to determine whether they show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein) and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced. In some embodiments, a population of different nucleic acids is partitioned into two or more different partitions. Each partition is representative of a different nucleic acid form, and a first partition (also referred to as a subsample) includes DNA with a cytosine modification in a greater proportion than a second subsample. Each partition is distinctly tagged. The first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The tagged nucleic acids are pooled together prior to sequencing. Sequence reads are obtained and analyzed, including to distinguish the first nucleobase from the second nucleobase in the DNA of the first subsample, in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine. The affinity agents can be antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target.

Examples of capture moieties contemplated herein include methyl binding domain (MBDs) and methyl binding proteins (MBPs) as described herein, including proteins such as MeCP2 and antibodies preferentially binding to 5-methylcytosine. Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4, RbAp48 and SANT domain peptides. Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (ThermoFisher Scientific)). Subsequently, additional partitioning may involve eluting fragments having different levels of methylation by adjusting the salt concentration in a solution with the methyl-binding domain and bound fragments. As salt concentration increases, fragments having greater methylation levels are eluted. In some instances, the final partitions are representative of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (ThermoFisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, at least 300 mM, at least 400 mM, at least 500 mM, at least 600 mM, at least 700 mM, at least 800 mM, at least 900 mM, at least 1000 mM, or at least 2000 mM. After such methylated nucleic acids are eluted, magnetic separation is once again used to separate higher levels of methylated nucleic acids from those with lower level of methylation. The elution and magnetic separation steps can repeat themselves to create various partitions such as a hypomethylated partition (representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent). The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition. For further details regarding portioning nucleic acid samples based on characteristics such as methylation, see WO2018/119452, which is incorporated herein by reference. In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

An exemplary method for molecular tag identification of MBD-bead partitioned libraries through NGS is as follows:

Physical partitioning of an extracted DNA sample (e.g., extracted blood plasma DNA from a human sample) using a methyl-binding domain protein-bead purification kit, saving all elutions from process for downstream processing.

Parallel application of differential molecular tags and NGS-enabling adapter sequences to each partition. For example, the hypermethylated, residual methylation (‘wash’), and hypomethylated partitions are ligated with NGS-adapters with molecular tags.

Re-combining all molecular tagged partitions, and subsequent amplification using adapter-specific DNA primer sequences.

Enrichment/hybridization of re-combined and amplified total library, targeting genomic regions of interest (e.g., cancer-specific genetic variants and differentially methylated regions).

Re-amplification of the enriched total DNA library, appending a sample tag. Different samples are pooled and assayed in multiplex on an NGS instrument.

Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nM to about 2500 mM NaCl. In one embodiment, the process results in three (3) partitions. Molecules are contacted with a solution at a first salt concentration and including a molecule including a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

The disclosure provides further methods for analyzing a population of nucleic acids in which at least some of the nucleic acids include one or more modified cytosine residues, such as 5-methylcytosine and any of the other modifications described previously. In these methods, after partitioning, the subsamples of nucleic acids are contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. The nucleic acids subjected to the procedure are then amplified with primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Such an analysis can be performed using the following exemplary procedure. After partitioning, methylated DNA is linked to Y-shaped adapters at both ends including primer binding sites and tags. The cytosines in the adapters are modified at the 5 position (e.g., 5-methylated). The modification of the adapters serves to protect the primer binding sites in a subsequent conversion step (e.g., bisulfite treatment, TAP conversion, or any other conversion that does not affect the modified cytosine but affects unmodified cytosine). After attachment of adapters, the DNA molecules are amplified. The amplification product is split into two aliquots for sequencing with and without conversion. The aliquot not subjected to conversion can be subjected to sequence analysis with or without further processing. The other aliquot is subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. Only primer binding sites protected by modification of cytosines can support amplification when contacted with primers specific for original primer binding sites. Thus, only original molecules and not copies from the first amplification are subjected to further amplification. The further amplified molecules are then subjected to sequence analysis. Sequences can then be compared from the two aliquots. As in the separation scheme discussed above, nucleic acid tags in adapters are not used to distinguish between methylated and unmethylated DNA but to distinguish nucleic acid molecules within the same partition.

Subjecting the First Subsample to a Procedure that Affects a First Nucleobase in the DNA Differently from a Second Nucleobase in the DNA of the First Subsample

Methods disclosed herein comprise a step of subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases includes mC and the other includes hmC.

In some embodiments, the procedure that affects a first nucleobase in the DNA differently from a second nucleobase in the DNA of the first subsample includes bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase includes one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion on a first subsample as described herein thus facilitates identifying positions containing mC or hmC using the sequence reads obtained from the first subsample. For an exemplary description of bisulfite conversion, see, e.g., Moss et al., Nat Commun. 2018; 9:5068.

In some embodiments, the procedure that affects a first nucleobase in the DNA differently from a second nucleobase in the DNA of the first subsample includes oxidative bisulfite (Ox-BS) conversion. In some embodiments, the procedure that affects a first nucleobase in the DNA differently from a second nucleobase in the DNA of the first subsample includes Tet-assisted bisulfite (TAB) conversion. In some embodiments, the procedure that affects a first nucleobase in the DNA differently from a second nucleobase in the DNA of the first subsample includes Tet-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes chemical-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes APOBEC-coupled epigenetic (ACE) conversion.

In some embodiments, procedure that affects a first nucleobase in the DNA differently from a second nucleobase in the DNA of the first subsample includes enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-BGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA of the first subsample includes separating DNA originally including the first nucleobase from DNA not originally including the first nucleobase.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

Techniques including methylated DNA immunoprecipitation (MeDIP) can be used to separate DNA containing modified bases such as mA from other DNA. See, e.g., Kumar et al., Frontiers Genet. 2018; 9:640; Greer et al., Cell 2015; 161:868-878. An antibody specific for mA is described in Sun et al., Bioessays 2015; 37:1155-62. Antibodies for various modified nucleobases, such as forms of thymine/uracil including halogenated forms such as 5-bromouracil, are commercially available. Various modified bases can also be detected based on alterations in their base-pairing specificity. For example, hypoxanthine is a modified form of adenine that can result from deamination and is read in sequencing as a G. See, e.g., U.S. Pat. No. 8,486,630; Brown, Genomes, 2nd Ed., John Wiley & Sons, Inc., New York, N.Y., 2002, chapter 14, “Mutation, Repair, and Recombination.”

### Example 1

### Process Overview

As described, several challenges are associated with small variant calling. Namely, the identification of non-ref loci and distinguishing true mutation from error are particular complex issues. In relation to sources of error, some examples include:
Recurrent error—Examples include alignment bias or sequencing artifact in low complexity regions. A training table, including using ZSCORE, can provide a mitigation strategy.
Random error—Examples include deamination or oxidation caused by DNA damage. Here, one may reduce random error by application of tier criteria (SNV) & LLR (Indel): complex criteria to filter random errors. Another approach involves use of a deamination filter (SNV): specific to filter deamination errors.

Generally, embodiments of the method described herein, simplify small variant calling to ZSCORE (recurrent error) and VSCORE (random error), while also reduce heuristic criteria, thereby providing improvements in accuracy, precision, while also reducing what would otherwise be intensive resource utilization.

### Example 2

### Technical Design

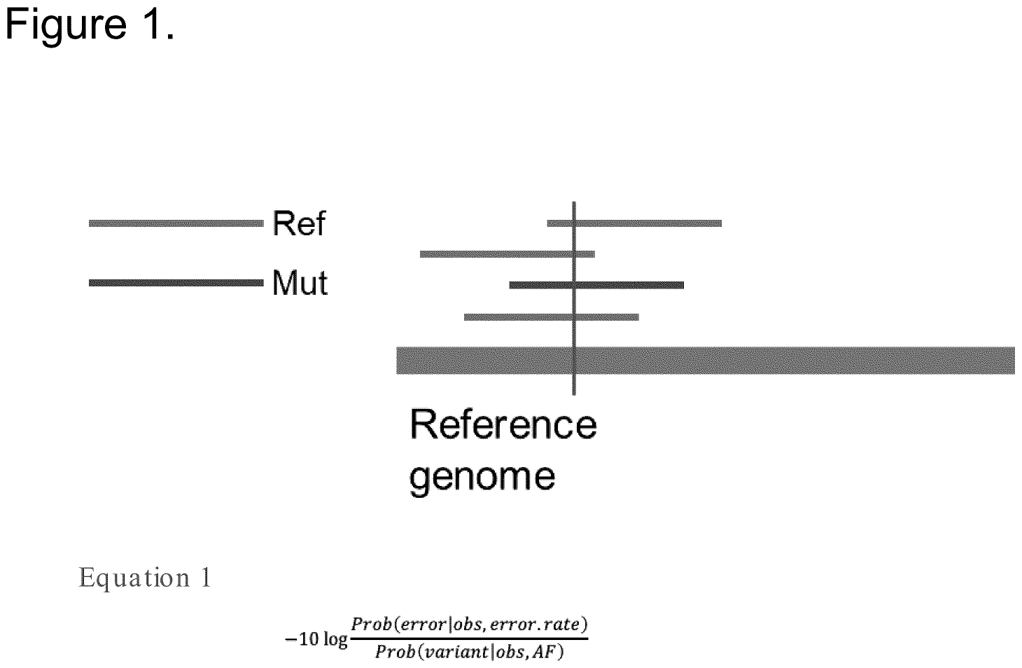

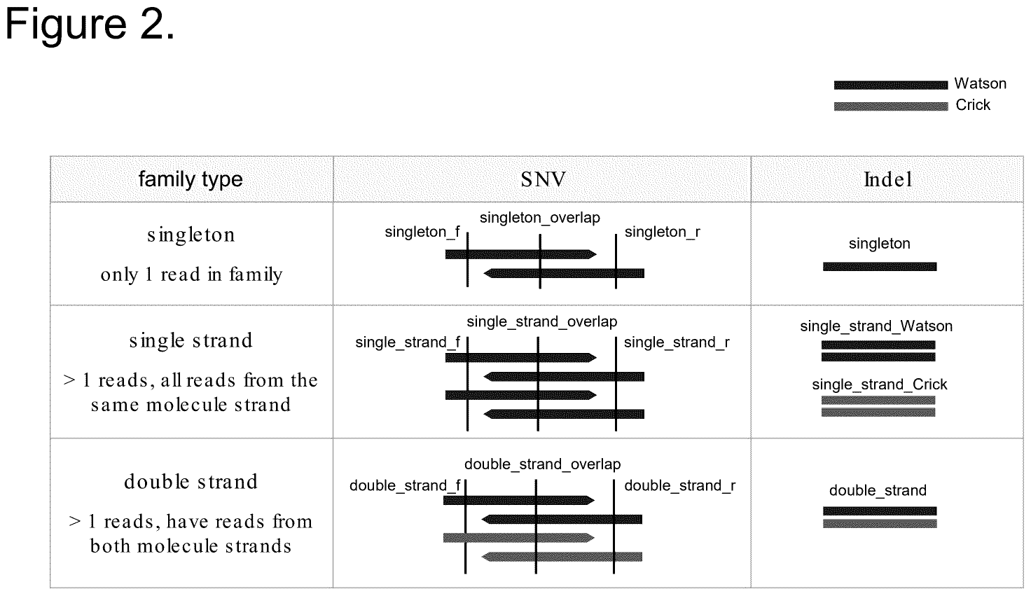

An exemplary process is described below:
Step 1: Family Grouping and Counting for Variant Candidate. Examples of family types are shown in FIG. 1, along with representations of SNVs and Indel.
Step 2: Variant Candidate Filtering Criteria are shown in Table 1 for SNV and Indel.

TABLE 1
Variant Candidate Filter Criteria

| | SNV | Indel |
|---|---|---|
| Main | Tiers | Log Likelihood |
| algorithm | | model |
| All genomic | DSO >= 1 and NS >= 3 | DS >= 1 and |
| positions | MAF >= 0.1% (0.2%) | FS >= 2 |
| | ZSCORE >= 8 | MAF >= 0.1% |
| | | LLR >= 10 |
| Hotspot | Cosmic CNT > 2 | EGFR, ERBB2, |
| positions | NS >= 3 and MAF >= 0.1% | MET |
| | and | DS >= 1 and |
| | ZSCORE >= 8 | FS >= 2 |
| | Hotspot variants | MAF >= 0.01% |
| | MAF >= 0.001% AND | LLR >= 6 |
| | NS >= 1 and DS >= 1 and | |
| | NS + SO >= 3 OR | |
| | NS >= 1 and DS >= 2 OR | |
| | NS >= 1 and DSO >= 1 and | |
| | NS + SO >= 2 | |

### Example 3

### SNV Calling

Random error characterization include a exemplary error rate˜family type+NT change. Here, one can take SNPs from healthy normal samples, remove potential germline, keep only mutants with AF<1% and estimate error rate using mutant count/total count with a 95% confidence interval.

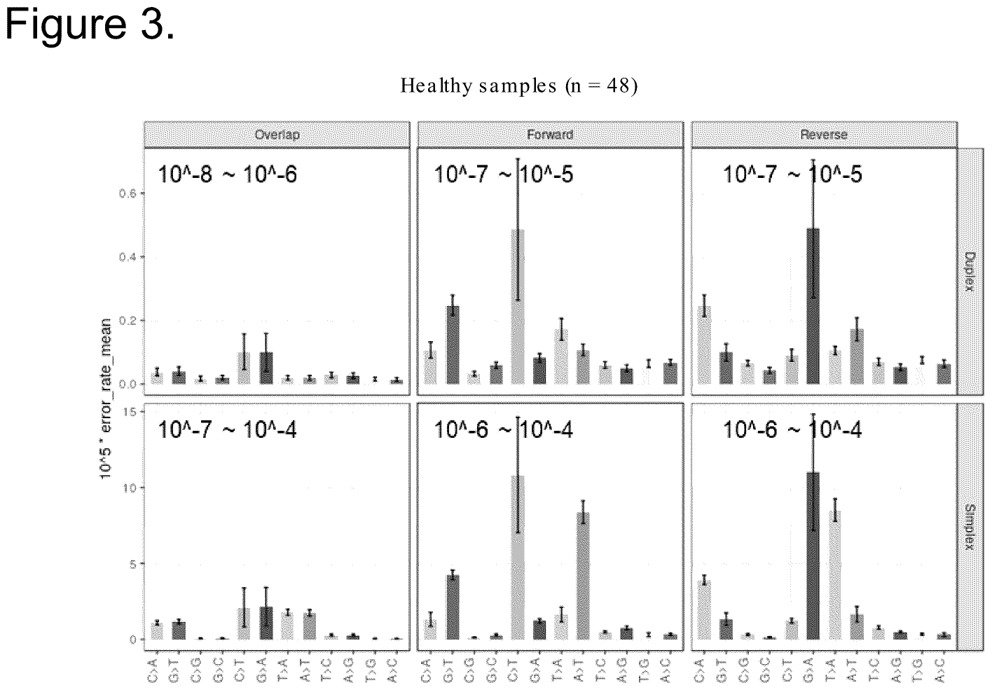

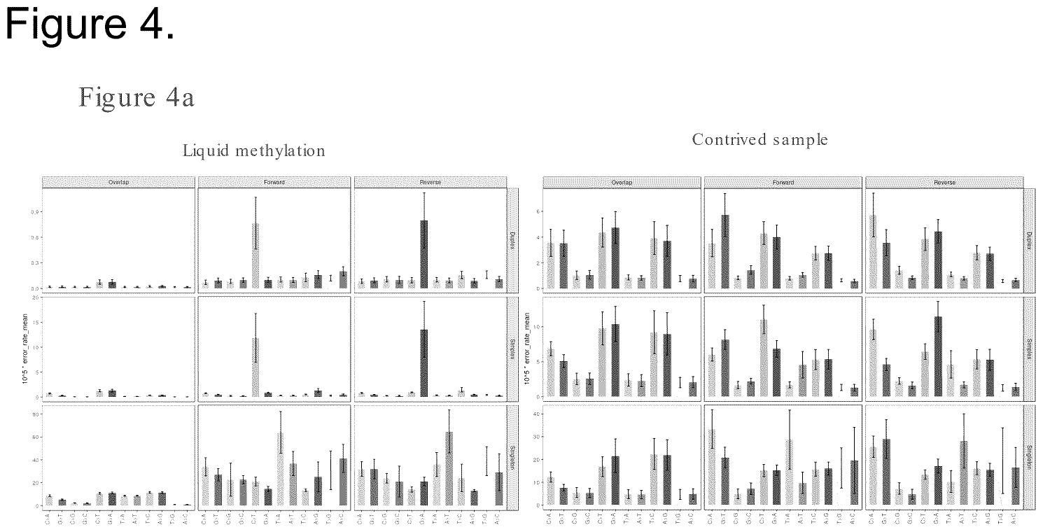

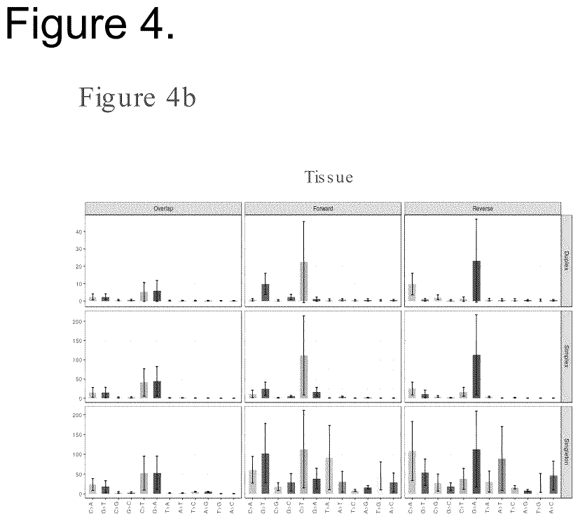

In FIG. 3, shown is error rate profile of different family types and NT changes Namely, an error profile may be characterized by Family Support+strand+NT change. For example, DS has lower error rate than SS, overlap has lower error rate than fwd and rev on most NT changes and DSO-lowest error rate. An additional source is DNA damage leading to strand bias in error rate. This includes deamination events (C:G→T:A) and oxidation (C:G→A:T).

### Example 4

### Error Rate Profiles of Different Panels and Materials

Different panels and materials possess different error rates. This includes for example:

For liquid biopsy LoB (n=276), samples were characterized by DS & SS with high C>T/G>A, low A>T/T>A.

Contrived samples (n=59) also have overlap is similar to fwd/rev, with DS not much lower than SS.

Tissue healthy samples (n=124) exhibited higher error rate than liquid including higher sample variation, high DNA damage related errors.

### Example 5

### Variant Score (VSCORE) Calculation

An exemplary calculation includes log likelihood ratio of error vs. true variant, including Equation 1. One can provide an independent calculation for different family types

### Example 6

### Indel Calling

Here, variant score calculation involves several input metrics including
Static conservative error rate on double strand (DS) and single strand (SS) molecules
   DS: 1e-6; SS_Watson & SS_Crick: 5e-5
Non-singleton observed AF
   (n_mut of DS+SS)/(n_total of DS+SS)
variant score calculation

Calculation of score on DS, SS_Watson, SS_Crick includes Equation 1.
variant score=score of DS+SS_Watson+SS_Crick

Further, an exemplary ZSCORE calculation uses baseline noise from healthy samples
Baseline noise per position in normal
   take all variants with family support >0 from N normal samples
   remove variants with family AF>=20% (likely germline)
   for each position, noise=sum(non-singleton AF)/N
Calculation of ZSCORE
   −log 10(1−pbinom (n_mut−1, n_total, noise))
   n_mut, n_total: DS+SS

### Example 7

### Main Criteria for Both SNV and Indel Calling

Based on the above, unified ZSCORE & VSCORE filtering as main criteria in both SNV and Indel calling allows one to keep molecule support and mutant allele fraction (MAF) filters, as well as utilization of additional filters to minimize changes, including the example provided in Table 2.

TABLE 2
Additional filter for SNV

| | SNV | Indel |
|---|---|---|
| Main algorithm | ZSCORE & VSCORE | |
| All genomic | ZSCORE >= 8 | ZSCORE >= 8 |
| positions | VSCORE >= 70 | VSCORE >= 50 |
| | NS >= 2 | DS >= 1 & FS >= 2 |
| | MAF >= 0.1% (0.2%) | MAF >= 0.05% |
| Hotspot positions | Pre-defined hotspot variants | EGFR. ERBB2, MET |
| | VSCORE + 40 >= 70 | VSCORE + 30 >= 50 |
| | MAF >= 0.001% | MAF >= 0.01% |
| Additional filters | deamination | read-level error |
| | read-level LLR | |
| | fragment end | |

### Example 8

### Additional SNV Fragment End Filter

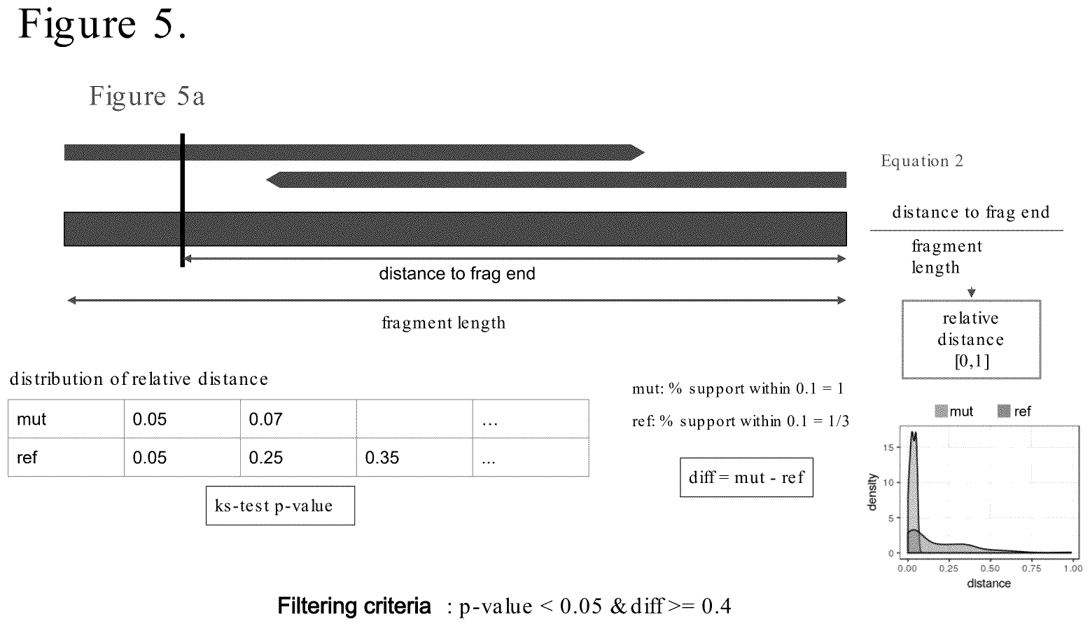

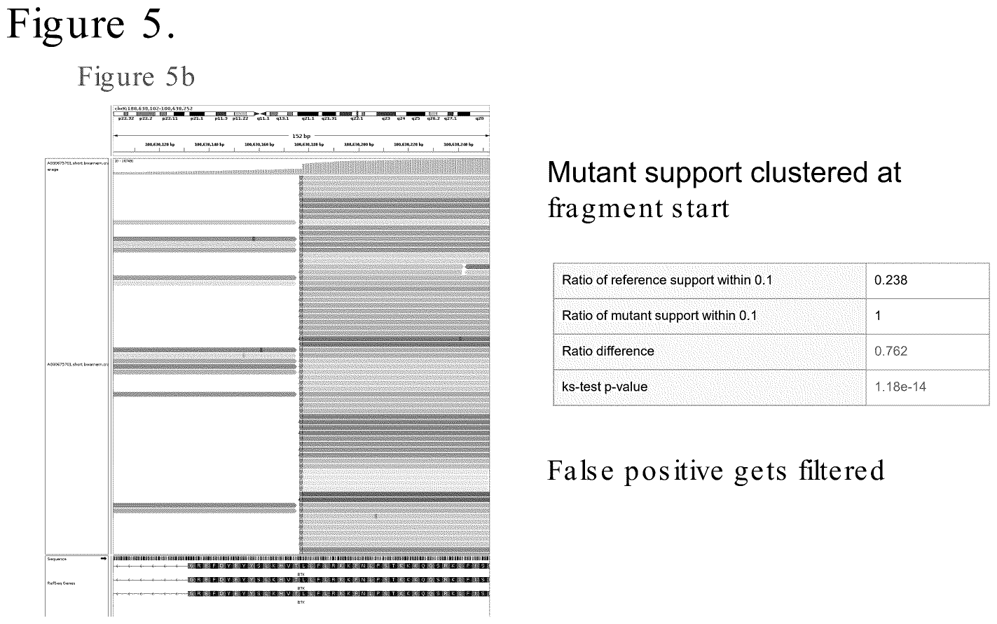

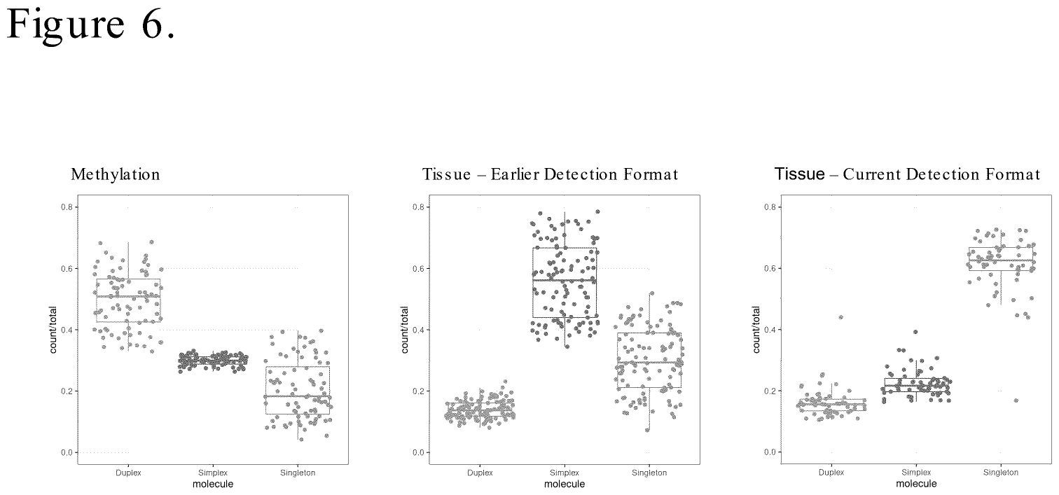

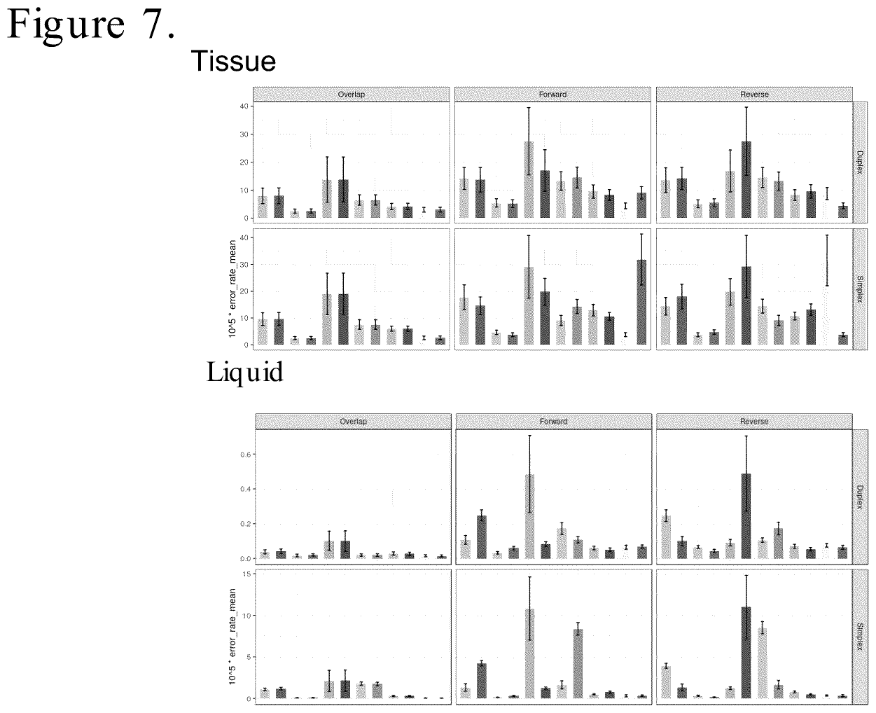

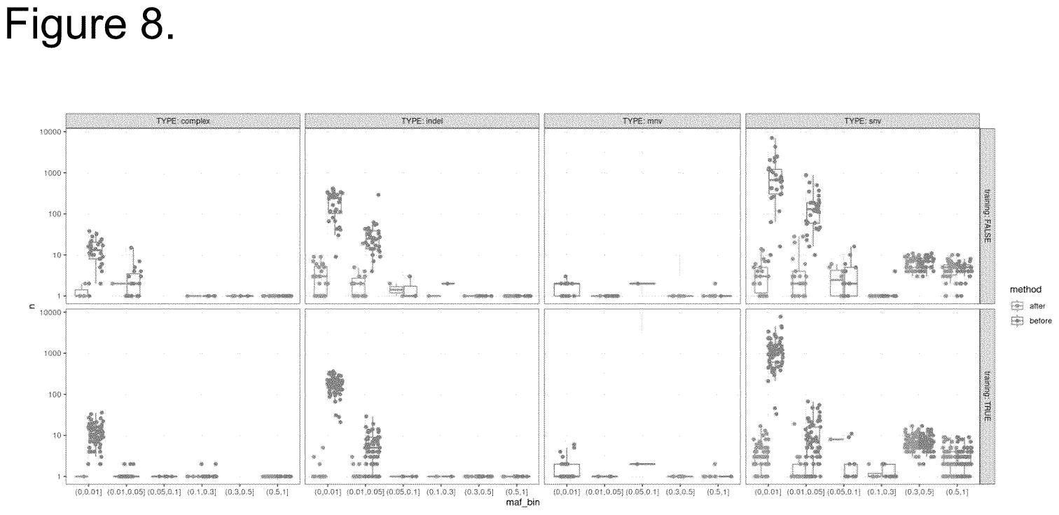

This additional filters includes a comparison of distribution of relative distance in mut and ref support, as depicted in FIG. 5a. As further shown in FIG. 5b, application of this filter removes FPs with mutant support clustered at fragment end/start

### Example 9

### Challenges in Different Source Materials

As described, there is lower diversity and lower double strand ratio in tissue than liquid. Even more challenging is the common scenario where one does not sequence to saturation in to maintain low sequencing reads, leading to high proportion of singletons. Here, a mitigation strategy is to add singletons into consideration for small variant calling, with the use of VSCORE and ZSCORE as described.

### VSCORE Training: Random Noise

SNV error rate˜mut_nt+molecule strand+read orientation
   Molecule strand
      Double strand (Watson+Crick)
      Single strand (Watson or Crick)
   Read orientation
      Forward/Reverse read only
      Overlap (Forward+Reverse)
Indel error rate˜molecule strand

### ZSCORE Training: Recurrent Noise

SNV: baseline noise for each position and mut_nt
Indel: baseline noise for each position

### Example 10

### Pre-Filter Criteria

Here, one can removes a majority of low MAF variants in normal samples as most residual variants are below 1%, which one can remove with MAF filter and identification of new error patterns for false positives >=1%.

### Example 11

### Exemplary Error Pattern

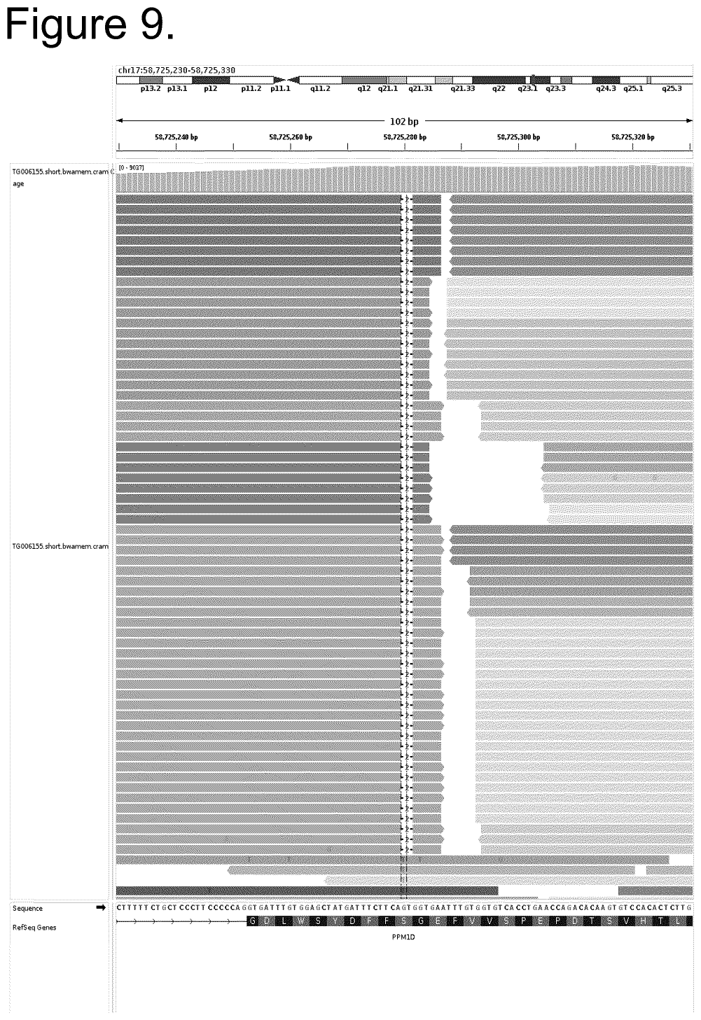

Using the described methods, an exemplary error pattern including Indel support enriched at fragment edge is shown in FIG. 9. Criteria include being variants present in both deletion and insertion, present in >1 samples in both normal training and normal from tumor-normal pairs. Thereafter, an additional filter process was implemented, including for example:
define bad distance
when Indel is <=10 bases from fragment start or end
count ratio of mutant support molecules with bad distance
fail Indel call if ratio >0.66

### Example 12

### Additional Exemplary Error Pattern

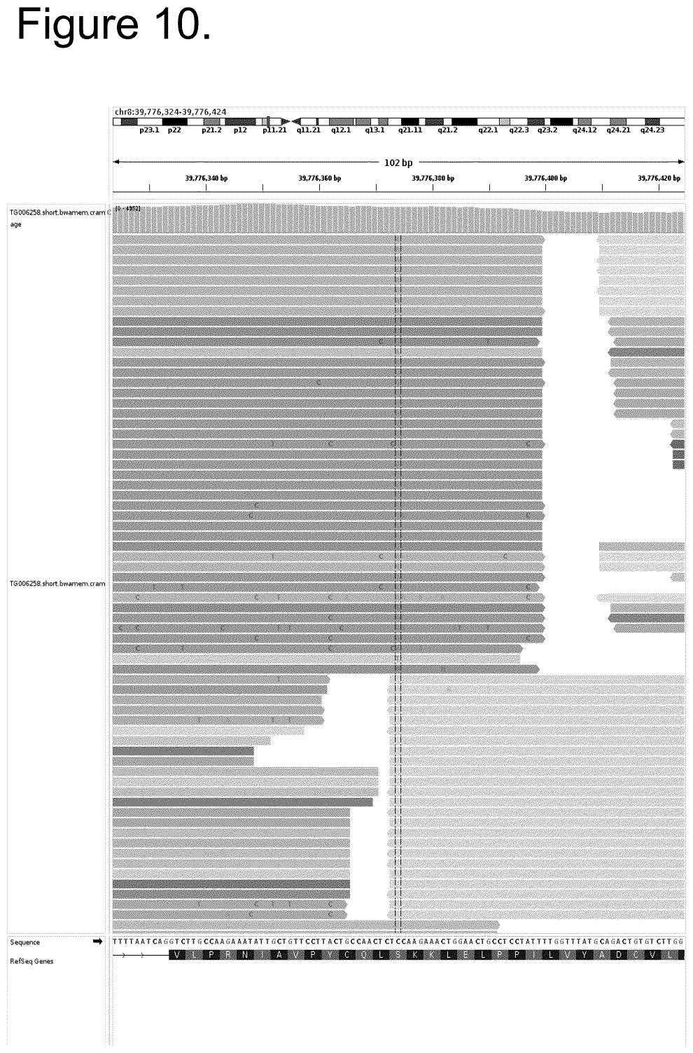

Here, an additional exemplary error pattern is shown in FIG. 10, including low diversity in mutant support due to family splitting, with criteria including low diversity in mutant support, being present in SNVs and Indels and present in >1 samples in both normal training and normal from tumor-normal pairs. Thereafter, an additional filter process was implemented:
mutant support <=10
create unique sets for mutant MK tags
start coordinate—{39776263, 39776264, 3977625}
end coordinate−{39776401}
UMI tag
get the size of each set
fail variant call if <2 sets with size >1

1. A method, comprising:
accessing sequence information for a plurality of sequence reads generated from a biological sample comprising nucleic acid molecules;
identifying a plurality of sequence reads based on a criterion;
categorizing each of the plurality of sequence reads into one or more family types;
determining an error rate for each of the one or more family types;
detecting the presence or absence of a genetic variant in the biological sample based on the determination of error rate of the categorized family type of the plurality of sequence reads.

2. The method of claim 1, wherein the error rate is a random error rate, recurrent error rate, or both.

3. The method of claim 1, wherein the criterion is an overlap criterion.

4. The method of claim 1, wherein the criterion is based on singleton, single strand, double strand criterion.

5. The method of claim 1, wherein the criterion is based on strand orientation.

6. The method of claim 1, comprising:
aligning the plurality of reads to a reference genome;
determining one or more loci based on the alignment of the plurality of reads.

7. The method of claim 6, wherein the detected genetic variant is at the one or more loci.

8. The method of claim 1, wherein the detected genetic variant is a SNV.

9. The method of claim 1, wherein the detected genetic variant is an insertion, deletion, and/or nucleic acid rearrangement.

10. The method of claim 2, wherein the random error rate is based on one or more of: number of the plurality of sequence reads categorized into one or more family types, strand orientation, strand bias, and nucleotide change.

11. The method of claim 2, wherein the recurrent error rate is based on baseline noise from reference samples.

12. The method of claim 11, wherein the reference samples are from normal subjects.

13. The method of claim 1, wherein the detected genetic variant is based on random error rate, recurrent error rate or both, and further wherein the random error rate is based on one or more of: number of the plurality of sequence reads categorized into one or more family types, strand orientation, strand bias, and nucleotide change and the recurrent error rate is based on baseline noise from reference samples.

14. The method of claim 1, wherein the detecting the presence or absence of a genetic variant further comprises a determination based on measurement of one or more of: deamination, read-level error, fragment position, genomic position, hotspot position, mutant allele fraction (MAF), and sequence read diversity.

15. The method of claim 1, comprising determining a predicted disease state based on the detected variant.

16. The method of claim 1, wherein
the error rate is a random error rate, recurrent error rate, or both,
the criterion is one or more of: an overlap criterion, a singleton, a single strand, a double strand criterion and a strand orientation, and
the detected genetic variant is a SNV, an insertion, deletion, and/or nucleic acid
rearrangement at one or more loci based on the alignment of the plurality of reads.

17. The method of claim 1, wherein
the error rate is a random error rate, recurrent error rate, or both,
the criterion is one or more of: an overlap criterion, a singleton, a single strand, a double strand criterion and a strand orientation,
the detected genetic variant is a SNV, an insertion, deletion, and/or nucleic acid
rearrangement at one or more loci based on the alignment of the plurality of reads, and further wherein
the random error rate is based on one or more of: number of the plurality of sequence reads categorized into one or more family types, strand orientation, strand bias, and nucleotide change, and
the recurrent error rate is based on baseline noise from reference samples.

18. The method of claim 1, wherein
the error rate is a random error rate, recurrent error rate, or both,
the criterion is one or more of: an overlap criterion, a singleton, a single strand, a double strand criterion and a strand orientation,
the detected genetic variant is a SNV, an insertion, deletion, and/or nucleic acid
rearrangement at one or more loci based on the alignment of the plurality of reads, and further wherein
the random error rate is based on one or more of: number of the plurality of sequence reads categorized into one or more family types, strand orientation, strand bias, and nucleotide change,
the recurrent error rate is based on baseline noise from reference samples, and further wherein
the detected genetic variant is based on random error rate, recurrent error rate or both, the random error rate is based on one or more of: number of the plurality of sequence reads categorized into one or more family types, strand orientation, strand bias, and nucleotide change and the recurrent error rate is based on baseline noise from reference samples, and
the detecting the presence or absence of a genetic variant further comprises a determination based on measurement of one or more of: deamination, read-level error, fragment position, genomic position, hotspot position, mutant allele fraction (MAF), and sequence read diversity.

19. A system configured to perform the method of claim 1.

20. A computer readable medium, comprising instructions for performing the method of claim 1.
